(19) United States
(12) Patent Application Publication
(10) Pub. No.: US 2005/0234448 A1
(43) Pub. Date: Oct. 20, 2005

(54) Implantable bone-lengthening device

(76) Inventor: James McCarthy, Merion, PA (US)

Correspondence Address: J. PETER FASSE;FISH & RICHARDSON P.C., 225 Franklin Street, Boston, MA, 02110, US

(21) Appl. No.: 11/084,518

(22) Filed: Mar. 18, 2005

Related U.S. Application Data
(60) Provisional application No. 60/554,776, filed on Mar. 19, 2004.

(51) Int. Cl. A61B 17/58

(52) U.S. Cl. 606/57

(57) ABSTRACT

The invention relates to implantable bone-lengthening devices that are not placed intra-medullarily, but are placed extramedullarily, i.e., outside of the bones, but under the skin. The devices do not require exposed hardware (that leads to infection) or skin and muscle penetration from the pins (that cause pain), and produce minimal scarring from pin sites because the devices are placed under the skin of a patient using minimally invasive techniques. The devices may be designed with smooth contours to enable implantation using minimally invasive techniques. The devices may be actuated using an actuator that is externally or internally powered. In the case of external power, the devices may be powered remotely through high frequency transmission of power through the skin. Also included are bone-lengthening devices having fluid reservoirs and conduits for storing and delivering therapeutic fluids to treatment sites.

### CROSS-REFERENCE TO RELATED APPLICATIONS

This application claims priority from U.S. Provisional Application Ser. No. 60/554,776, filed on Mar. 19, 2004, the contents of which are incorporated herein by reference in their entirety.

### TECHNICAL FIELD

This invention relates to bone-lengthening devices and related systems and methods.

### BACKGROUND

Children and adults sometimes require lengthening of one or more of their bones. Typically, this is done using one of many types of external fixation devices that use pins that penetrate the skin, muscle, and bone. Although these are effective, numerous complications often occur, such as pain from the pins that can lead to decreased joint range, pin site infections and osteomyelitis. Patients are required to clean the pins twice a day, which can be painful, especially in children. Other complications include fracture after the fixation device is removed, as well as bowing, and scars at the pin sites. The bowing is due to the distance between where the force is applied and the bone, resulting in a torque on the bone instead of a linear force.

In an effort to decrease these problems, several implantable intramedullary (IM) lengthening devices have been proposed, including the Albizzia nail, the Intramedullary Skeletal Kinetic Distractor, the Munich Nail, and the Lisa/Phenix Prothesis.

However, there are difficulties in using intramedullary lengthening devices, especially in children. These difficulties often involve the growth plates both proximally and distally in both the femur and the tibia. For example, antegrade cannulization, i.e., inserting a tube into a hollow inside of a bone, of the femur not only disrupts the greater trochanter growth plate, but can lead to avascular necrosis of the femoral head in children and certainly can disrupt growth quite significantly in the tibia, if the children's growth plates are open. Another issue is that intramedullary channels in children have small diameters that often do not leave sufficient room for intramedullary lengthening devices.

Thus, external fixation devices continue to be used to treat children. However, as noted, these external fixation devices have multiple complications and difficulties and create a painful process for children, not only after surgery, but also throughout the length of device placement.

Various bones can require lengthening. The long bones in the legs, and sometimes the arms are the bones most typically lengthened. The backbone can also be lengthened or straightened. For example, rib expanders are used primarily for children with infantile scoliosis that does not respond to conservative therapy, or other young children with severe kyphotic or scoliotic deformities and possible pulmonary compromise that are too young to fuse. Currently, rib expanders or “growing rods” are surgically implanted and then the child is taken back to surgery every 1 to 6 months to expand the rod.

### SUMMARY

This invention relates, in part, to implantable bone-lengthening devices that are not placed intra-medullarily, but are placed extramedullarily, i.e., outside of the bones. The devices do not require exposed hardware (that leads to infection) and muscle penetration from the pins (that cause pain), and do not leave dramatic scars at pin sites, because the device is placed under the skin of a patient using minimally invasive techniques. The devices are typically designed with smooth contours to enable implantation using minimally invasive techniques. The devices can be actuated using an actuator that is externally or internally powered. In the case of external power, the devices may be powered remotely through transmission of power through the skin.

The invention further relates, in part, to bone-lengthening devices having a reservoir and a pump integrated into the devices to administer growth factors, antibiotics, pain medication, or other desired therapeutic agents at the appropriate time(s).

In one aspect, the invention features extramedullary elongation devices for lengthening one or more bones, the devices to be implanted adjacent to the bone and under the skin of a patient using minimally invasive techniques. The devices include a frame having smooth edges and an end with a smooth contour, a first plate attached to the frame and configured to be secured to the bone, the first plate having smooth edges, a second plate configured to be secured to the bone, the second plate having smooth edges, a rod (which may be enclosed by the frame) linked to the first plate, an actuator secured either to the rod or the second plate, and a block secured to the second plate, the block linked to the rod such that actuation of the actuator results in displacement of the second plate relative to the first plate. The device has a cross-sectional diameter of no more than about 3.0 cm.

The term “adjacent,” when used to describe the location of the device with respect to a bone means that the device is located under the skin and typically under much or all of the muscle overlying a bone. The device can, but need not, directly contact the bone.

The term “smooth” when used to describe components of the device means that the exposed or outer surfaces of the components when the device is assembled present no sharp corners or edges. In addition, a smooth surface inhibits cells or tissues from adhering to the device. The term “linked” when used to describe the connection between the rod and other components of the device means that the rod and the component are connected, but does not require that they be rigidly connected; the rod may, for example, rotate within or slide through a component to which it is linked. No direct contact between the rod and the particular component of the device to which it is linked is required; the rod may, for example, be linked to the first or second plate via an actuator.

In a second aspect, the invention features extramedullary elongation devices for lengthening one or more bones, the devices to be implanted adjacent the bone and under the skin of a patient using minimally invasive techniques. The devices include a first plate, a second plate, a rod linked to the first plate, an actuator secured either to the rod or the second plate, and a block secured to the second plate, the block linked to the rod such that actuation of the actuator results in displacement of the second plate relative to the first plate. Screws are provided that secure the first plate and second plate to the bone. The screws are no more than about 50 mm in length. The first plate may have smooth edges. The second plate may have smooth edges. Embodiments may further have a frame attached to the first plate and, in some embodiments, enclosing or partially enclosing the rod, and the frame may have smooth edges and an end with a smooth contour.

In yet another aspect, elongation devices are provided that are capable of storing and delivering therapeutic agents or fluids to the gap between portions of the bone being elongated. The device includes a first member configured to be secured to the bone and a second member configured to be secured to the bone. The first and second members are operably connected such that, upon actuating an actuator, the second member displaces or moves relative to the first member. The device includes a reservoir for storing a therapeutic fluid, a conduit connected to the reservoir for delivering the therapeutic fluid to a desired location, and optionally a pump capable of pumping fluid from the reservoir through the conduit. The pump can be actuated by the actuator that acts to separate the first and second members such that fluid is pumped through the conduit upon actuating the device and spreading apart portions of bone being elongated. Alternatively, the pump can have its own actuator.

Embodiments may include one or more of the following. The rod can be enclosed by the frame. The rod can have any desired cross-sectional shape (e.g., round, oval, square, hexagonal). The cross-section of the device can be about 2 cm across (e.g., 2 cm in diameter), and the device can be about 4 to 8 cm long, e.g., 5, 6, or 7 cm. The device (and rod) can be either of uniform or non-uniform diameter or cross-sectional area. The rod can be threaded, and the block can have a threaded hole configured to be threaded on the rod upon actuation of the actuator. The rod can have slots configured to accept teeth from a gear of the actuator.

The actuator can be secured to the threaded rod and the first plate, the first plate can be attached to the frame, actuation of the actuator rotates the rod, and the second plate is slideably engaged with the frame. The actuator can be attached to the second plate, the first plate can be attached to the frame, and the second plate can be slideably engaged with the frame. The actuator can be powered by a battery, spring, or by a smart metal (such as Terfenol-D®)or other expandable device. The actuator can be a linear positioning stage. The actuator can be a bi-directional motor and the elongation device can further include a controller that controls the actuator. The controller can be configured for placement between the skin and the bone. The controller can be connected to the elongation device. In some cases, the controller can be located externally to the skin of the patient and the controller can transmit power and/or control signals via radio frequency to the motor.

In some cases, the device further can include a manual crank that is located externally to the skin of the patient, wherein the manual crank is configured to be mechanically connected to the rod through an opening in the skin such that rotation of the crank rotates or otherwise extends the rod.

The first and second plates can be configured to be secured to a long bone such as a tibia or a femur, and the translation of the second plate relative to the first plate results in elongation of the long bone. The first plate can be configured to be secured to a first rib bone or vertebra and the second plate can be configured to be secured to a second rib bone or vertebra, and the translation of the second plate relative to the first plate results in elongation or straightening of a spinal column. The actuator can be configured such that the rate of translation of the second plate relative to the first plate is from about 0.25 millimeter to 2 millimeters per day. The plate may be movable in either direction, i.e., to either increase or reduce the space between the first and second plates.

The first and second plates each can have two or more holes, and the plates can be secured to the bone using two or more screws. The screws may be of a length such as to extend through the plates and into the bone without extending completely through the bone. The screws may be no more than about 40 mm in length. The screws may be between about 25-50 mm, e.g., about 25-40 mm, in length. The screws may be threaded for substantially their entire length. The holes in the plates may be threaded to receive the threads of the screws and lock the plates to the screws. The screws can be threaded over about 50% of their length. The screws may have two threaded portions, the first at a first end of the screw and threaded over about 50% of the screw length for insertion into the bone and the second at an opposite end of the screw and threaded over about 10-25% of the screw length for locking the screw into the plate. The second threaded portion may have finer threads than the first portion.

All or parts of the device may be coated with a coating, such as a biologically inert or biologically compatible coating. Such a coating can enable the device, or components of the device, to be made from materials which are not biologically compatible or inert, wherein the coating would render the device compatible. The coatings can include plastics or polymers, ceramics, metallic platings, or other suitably inert coatings. Examples of biologically inert or biologically compatible coatings include parylene, polyethylene glycol (PEG) (e.g., asymmetric carboxylated PEG), ultrananocrystalline diamond (UNCD), tetrafluoroethylene (ETFE), polytetrafluoroethylene (PTFE), and perfluoro-alkoxy (PFA).

The therapeutic fluid may include bone growth factors or other bone growth promoting compositions, pain medication, antibiotics, antiviral drugs, or any combination thereof. The reservoir may be implantable. The conduit may deliver fluid to the bone itself, to the area surrounding the bone, or both. The conduit may deliver fluid to the regions in which portions of the device extend through muscle and/or skin, for example, to provide pain medication and/or antibiotics to such areas. The amount of fluid delivered may be a function of the degree of movement of the first member relative to the second member. The reservoir can be connected to a pump actuator capable of being actuated separately from the actuator. The device can further include a pump controller that controls the pump actuator. In some cases, the pump controller can be located externally to the skin of the patient, the reservoir can be implanted, and the controller can transmit power and control signals via radio frequency to the pump actuator.

In another aspect, the invention features methods for elongating a long bone of a patient. The methods include performing an osteotomy of the bone, implanting a new elongation device in accordance with any of the aspects and embodiments provided herein using a minimally invasive technique by making an incision in the skin, inserting the end of the frame into the incision, and then sliding the remainder of the device through the incision, securing the device to the bone (for example, by securing the first plate to the bone on one side of the osteomy and securing the second plate to the bone on the opposite side of the osteomy), and closing the incision.

In another aspect, the invention features methods for straightening a spine of a patient. The methods include creating an incision in the skin over a rib cage of the patient, implanting an elongation device in accordance with any of the aspects or embodiments provided herein using a minimally invasive technique by inserting the end of the frame into the incision, and then sliding the remainder of the device through the incision, securing the device to the first and second ribs (e.g., by securing the first plate to a first rib and securing the second plate to a second rib), and closing the incision in the patient.

In other aspects, the invention features methods for elongating a long bone or for straightening a spine of a patient. The methods include filling the reservoir of a new elongation device, in accordance with any of the aspects and embodiments provided herein that include a reservoir, with a therapeutic fluid; performing an osteotomy of the bone; securing the first member of the device to a first portion of the bone; securing the second member of the device to a second portion of the bone; directing the conduit to a treatment area; actuating the device to separate the first and second portions of the bone; and delivering therapeutic fluid to the treatment area. The straightening of the spine includes filling the reservoir of a new elongation device, in accordance with any of the aspects and embodiments provided herein that include a reservoir, with a therapeutic fluid; securing the device to first and second ribs; directing the conduit to a treatment area; actuating the device to expand the ribs; and delivering therapeutic fluid to the treatment area.

Embodiments may include one or more of the following. The elongation device can be inserted through a 2-4 cm incision. The first and second plates each can have two or more holes, and securing the device further can include drilling four or more holes in the long bone or ribs and placing screws through the holes in the plates into the bone or ribs. The first and second plates of the device can be configured to be secured to a long bone such as a tibia or a femur, and the translation of the second plate relative to the first plate results in elongation of the long bone. The first and second plates of the device can be configured to be secured to first and second ribs, respectively, and the translation of the second plate relative to the first plate results in expansion of the ribs. The first and second ribs may be adjacent to each other or may be separated by one or more ribs. The method can further include actuating the actuator. The actuator can be configured such that the rate of translation of the second plate relative to the first plate is from about 0.25 millimeter to 2 millimeters per day.

The block can include a threaded hole configured to be threaded on the threaded rod upon actuation of the actuator, wherein the device further can include a manual crank that is located externally to the skin of the patient, and wherein the manual crank can be mechanically connected to the rod such that rotation of the crank rotates or otherwise extends the rod. The method can further include removing a cap on the end of the frame, inserting a mechanical linkage of the manual crank into the patient, connecting the mechanical linkage to the rod, and turning the manual crank to translate the second plate relative to the first plate.

In elongation devices that include a fluid reservoir, the reservoir may be filled prior to implanting the device, or may be filled subsequent to implanting the device, for example by piercing a reservoir septum, e.g., made of rubber or plastic, with a needle and injecting therapeutic fluid into the reservoir. The reservoir can include a system for signaling when it is empty or nearly empty, e.g., by means of a wireless signal to a receiver located outside the body. The therapeutic fluid may be delivered from the reservoir to the space between the first and second plates, e.g., to a therapeutic area, by means of a pump. The therapeutic fluid may include bone growth factors or other bone growth promoting compositions, pain medication, antibiotics, antiviral drugs, or any combination thereof.

The therapeutic fluid may be delivered simultaneously with the actuating of the elongation device. The treatment area may be a bone being elongated, for example, the portions of bone exposed by the osteomy, may be muscle and/or skin being penetrated by at least a portion of the device, or any combination of these. The reservoir may be implantable.

These and other embodiments may have one or more of the following advantages. In contrast to external fixation devices that can generate pin site infections and more serious osteomyelitis, the extramedullary devices, in accordance with this invention, are implantable and therefore decrease the chance of infection after surgery. Also in comparison with external fixation devices, the extramedullary devices are placed closer to the bone so a problem of bending the bone away from its natural direction is lessened.

The extramedullary devices also allow for gradual displacement and a controlled basis for expanding the spine that reduces the frequency of surgery. Conventional rib expander devices currently need to be re-lengthened manually and the patient needs to undergo an additional surgery for each lengthening. Thus, the extramedullary devices can be beneficial for children with infantile scoliosis that does not respond to conservative therapy, and other young children with severe kyphotic or scoliotic deformities and possible pulmonary compromise that are too young to fuse.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a perspective view of an extramedullary bone-lengthening device that is lengthening a bone.

FIG. 2A is a side view of an extramedullary bone-lengthening device.

FIG. 2B is a top view of the extramedullary bone-lengthening device.

FIGS. 2C and 2D are end views of the extramedullary bone-lengthening device.

FIG. 2E is a view of an extramedullary bone-lengthening device with external power.

FIG. 2F is a view of an extramedullary bone-lengthening device with internal power and an external controller.

FIG. 2G is a view of an extramedullary bone-lengthening device with external power and a reservoir.

FIG. 3A is a diagrammatic view of an extramedullary bone-lengthening device of being implanted next to a bone.

FIG. 3B is a diagrammatic view of the extramedullary bone-lengthening device after implantation.

FIG. 4A is a diagrammatic view of an extramedullary bone-lengthening device that is secured to a tibia bone.

FIG. 4B is a diagrammatic view of the extramedullary bone-lengthening device after elongating the tibia bone of FIG. 4A.

FIG. 5 is a diagrammatic view of a manual crank used to actuate an extramedullary bone-lengthening device.

FIG. 6A is a diagrammatic view of an extramedullary bone-lengthening device implanted on ribs of a spine.

FIG. 6B is a diagrammatic view of the extramedullary bone-lengthening device after elongating the spine of FIG. 6A.

Like reference symbols in the various drawings indicate like elements.

### DETAILED DESCRIPTION

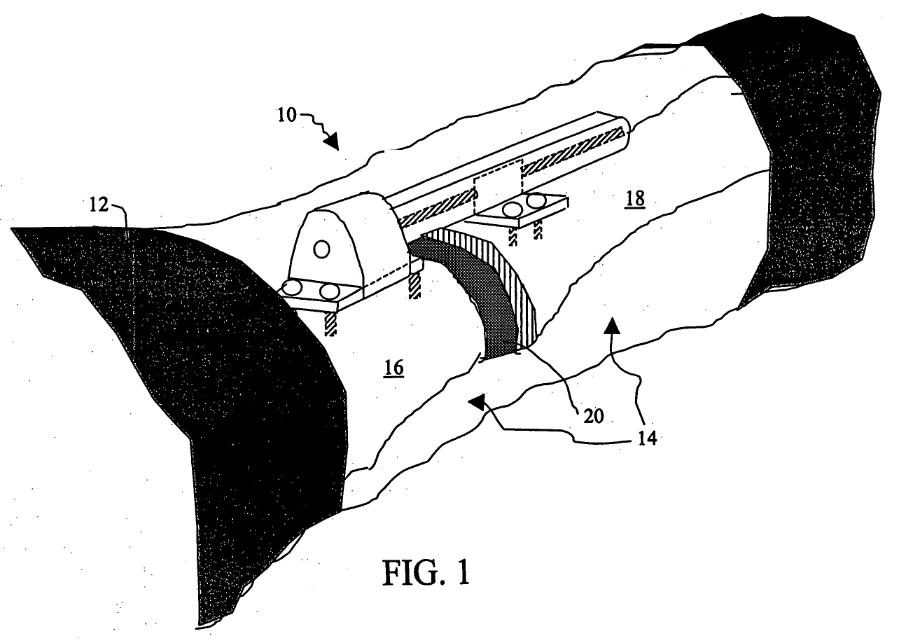

FIG. 1 shows an extramedullary bone-lengthening device 10 implanted under the skin 12 and adjacent to a bone 14 of a patient for lengthening the bone 14 after an osteotomy 20. The osteotomy 20 separates the bone 14 into two parts 16 and 18. The device 10 has a fixed part that is screwed to the bone part 16 and a moving part that is screwed to the bone part 18. An actuator is used to move the parts 16 and 18 apart under precise control over time so that bone tissue can grow in the osteotomy 20 and the bone 14 is elongated. The actuator is capable of moving the parts 16 and 18 apart at a rate of translation from about 0.25 millimeter to 2 millimeters per day. Either end can be the “fixed” part.

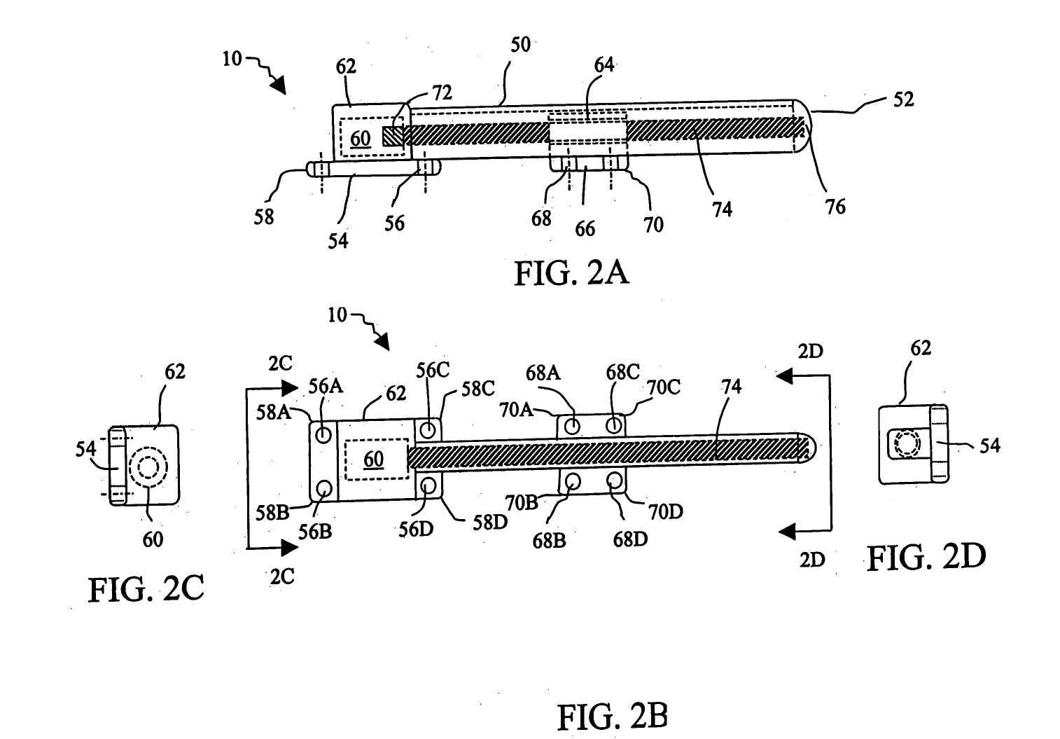

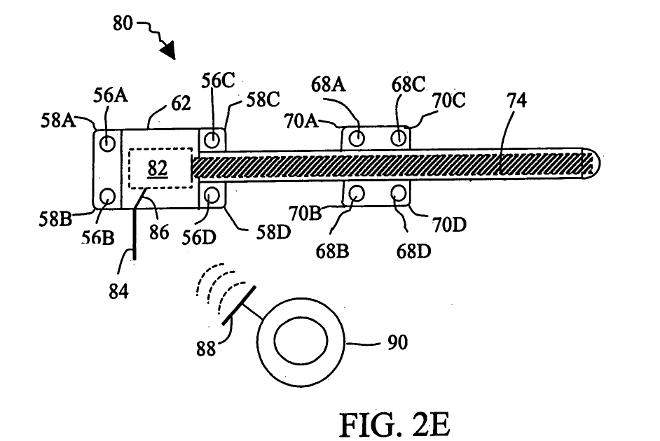

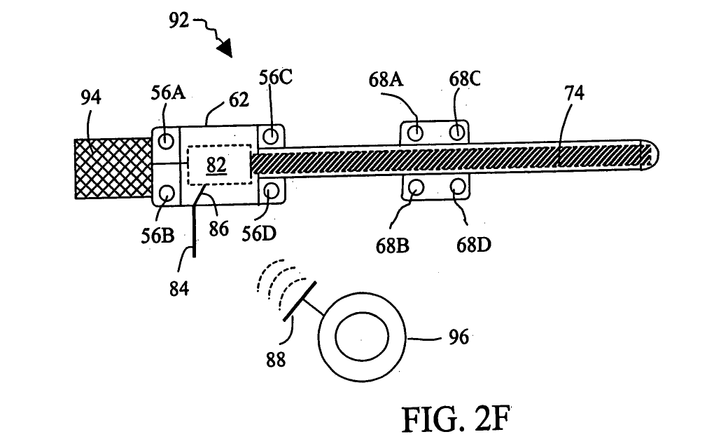

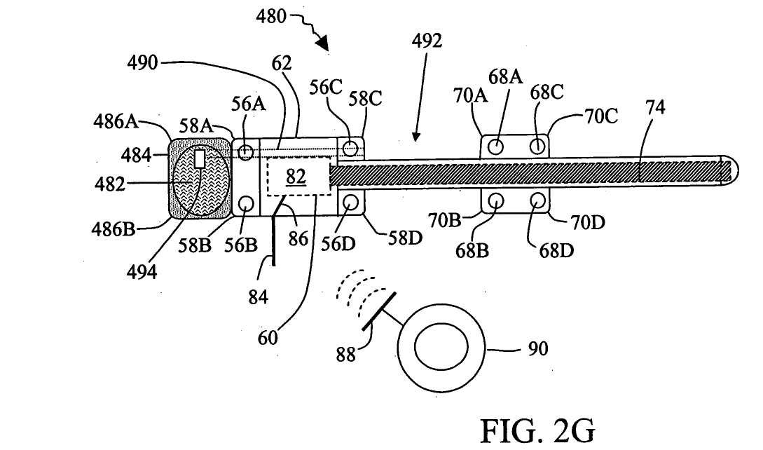

As shown in FIGS. 2A, 2B, 2C, and 2D, the device 10 includes a hollow frame 50 with an end cap 52 having a smooth, rounded contour. In some examples, the end cap 52 is removable for access to the frame 50. The device 10 also includes a plate 54 with clearance holes 56A, 56B, 56C, and 56D that enable mechanical fasteners to be secured to the bone beneath the plate 54. The plate 54 also has smooth, rounded edges 58A, 58B, 58C, and 58D. The device 10 also includes an actuator 60 that is secured to a housing 62, and is capable of rotating a shaft. The mechanical fasteners can be, for example, screws, bolts, rivets, nails, tacks, or nuts. Alternative fastening means such as adhesives can also be used. The parts of the device 10 can be made of materials to which biological tissues tend not to adhere such that the device 10 can be removed with minimal trauma to the patient after therapy is finished. Such materials include metals such as surgical grade stainless steel, titanium, and titanium alloys. Other metals or other materials, for example, ceramics, plastics, or carbon fiber materials can also be used, optionally with biologically inert coatings. Such coatings can be formed from various ceramics or inert plastics, such as polyurethanes, polyethylenes, polycarbonates, or mixtures and copolymers thereof.

In some examples, the actuator 60 is a bi-directional electromagnetic motor. Such motors include direct current (DC) rotary motors (brush or brushless), alternating current (AC) rotary motors, and rotary stepper motors. In the case of DC or AC rotary motors, rotary encoders on the shaft 72 are used for position feedback for the position control of the rod 74. In other examples, the actuator 60 rotates a shaft using potential energy of a spring mechanism (not shown).

The housing 62 is secured to the plate 54. The device 10 also includes a block 64 that is secured to a plate 66. The block 64 can have sealed ball-bushing guides (not shown) to enable the block 64 to smoothly slide along the frame 50. The plate 66 has screw clearance holes 68A, 68B, 68C, and 68D, which enable screws to be secured into the bone beneath the plate 66. The plate 66 has smooth, rounded edges 70A, 70B, 70C, and 70D. The actuator 60 rotates a shaft 72 that is secured to a threaded rod 74. The threaded rod 74 has an end 76 that, in some examples, has a socket cavity (not shown) for manual turning of the rod 74.

As shown in FIG. 2E, device 80 includes an actuator 60 powered by a bi-directional electromagnetic motor 82. In this arrangement, power can be transmitted to the motor 82 remotely through the skin 12. This avoids having to implant a power source such as a battery with the device 80 to provide power to actuate the motor 82.

Power can be transmitted to the motor 82 using a variety of known techniques used to power intramedullary and other devices. See, for instance, Baumgart et al, “A Fully Implantable Motorized Intramedullary Nail for Limb Lengthening and Bone Transport”, Clinical Orthopaedics and Related Research, 342: 135-143 (1997). For example, a reception antenna 84 with a diameter of approximately 2 cm and a thickness of approximately 4 mm can be implanted and connected to the motor 82 by an insulated thin flexible wire 86. The patient moves about normally during the day, and the motor 82 is actuated at night. The energy supply unit 88 is placed by the bed, and the transmitter 90 is taped to the skin like an electrocardiography electrode, over the reception antenna 84. The daily displacement can be programmed to take place evenly throughout a range to 600 micro intervals (e.g., 0.0008-0.003 mm) at a rate of translation from about 0.25 to 2 millimeter (mm) per day, e.g., 1 mm per day. Such lengthening can occur continuously throughout all or part of each day, in a single step, or may be accomplished in a series of discrete lengthening steps, for example, 1/100 mm per time, one hundred times per day, 1/10 mm per time, ten times per day, or ¼ mm per time, four times per day.

FIG. 2F shows a device 92 that includes an electromagnetic motor 82. A battery 94 is implanted with the device 92. This battery 94 is similar to a battery used for a pacemaker or a Baclofen pump. Actuation of the motor 82 is controlled via an RF transmission to the reception antenna 84. A controller 96 sends the RF transmission with control signals to the motor 82 via the reception antenna 84. Alternately, the motor can be controlled with a pre-programmed microchip that is implanted with the device 92. The microchip may instruct the motor to perform lengthening steps as described above in terms of degree of movement of one plate relative to the other (e.g., 1/100 mm per time, one hundred times per day, 1/10 mm per time, ten times per day, or ¼ mm per time, four times per day). The microchip may instruct the motor to run for set durations (e.g., on for 10 seconds, off for 50 minutes), where the time the motor runs is correlatable with the degree of movement of one plate relative to the other.

The motor 82 is typically actuated in one direction that increases the separation between the bone parts 16, 18. In some situations however, it is useful therapeutically to decrease the separation. This can occur, for instance, when the bone has not grown to close the separation sufficiently while the device 92 separates the bone parts. In this instance, a surgeon may decide to decrease the separation to enable the bone tissue to recover. The device 92 is then actuated in an opposite direction to bring the bone parts 16, 18 closer together. At a later stage, the surgeon may decide to actuate the device 92 in the normal direction again, to continue lengthening.

In alternate embodiments, devices similar to the device 10 may be secured to the bone without using screws. In these embodiments, the devices are secured to the bone using alternate means such as clamps or adhesive.

In alternate embodiments, linear drive motors may be used to displace the plate 66 away from the plate 54. In such embodiments, the linear drive motors move the plate 66 relative to the plate 54 as a positioning stage on ball bushing guides on a single or multiple linear rails inside the frame. Alternative power sources include springs and other materials or devices that convert one type of energy, such as electricity, into another, such as mechanical motion, such as smart metals.

FIG. 2G shows a device 480 that includes a reservoir 482 contained within a reservoir chamber 484. Reservoir chamber 484 has smooth, rounded edges 486A and 486B. The reservoir may be a chamber formed within one of the plates such that plate makes up the reservoir chamber. Conduit 490 is connected to reservoir 482 and extends to the gap or space 492 between the first and second plates, where the opening in the bone would reside. The conduit may be a tube or channel. The conduit may be the opening from the reservoir to the space between the first and second plates. The conduit can also be a tube that the surgeon inserts into the space between the bones. The reservoir serves as a chamber for storage of a therapeutic fluid, e.g., a desired drug, for subsequent delivery to the gap in the bone being lengthened and the surrounding area. Such fluid could then be pumped from the reservoir through the conduit to the gap in the bone. For example, it may be desired to provide bone growth stimulating factors (e.g., bone morphogenic protein (BMP), insulin-like growth factor (IGF)) or drugs that promote bone growth (e.g., teriparatide and/or sodium fluoride). Other desired fluids may be so delivered to the gap in the bone, including pain medications (including short and long lasting opiates, such as morphine, hydromorphine, codeine, hydrocodone, oxycontin, meperidine, fentanyl, MS contin, lavorphanol, methadone, propoxyphene, oramorph SR and/or oxymorphone; and/or local anesthetics, such as lidocane, mexiletine, and.or flexaininide), anti-inflammatory drugs (including steroidal anti-inflammatories, such as prednisone and/or dexamethasone), antibiotics (including ample spectrum penicillins such as amoxicillin, penicillins, beta lactamase inhibitors, cephalosporins, macrolids, lincosamines, quinolones, fluoroquinolones, carbepenems, monobactams, aminoglycosides, glycopeptides, tetracyclines, sulfonamides, rifampins, oxazolidonone, streptogramins, and/or other antibiotics) or antivirals (e.g., vidarabine, acyclovir, gancyclovir, protease inhibitors, ribavirin, and/or interferon). The therapeutic fluid may be a liquid, a gel, or a paste, and may comprise a sustained release product that slowly dissolves or releases a drug or other therapeutic substance over several hours, days, weeks, or months. The reservoir could be refillable, e.g., could have an inlet port that may be covered by a septum that would seal fluid in but could be pierced by a needle to refill with fluid. The reservoir could be filled either before implantation, after implantation, or both.

A pump 494, e.g., a micropump, could be actuated by actuator 60. The power to drive the pump can be provided by the same power source used to power the activator, e.g. a power source that delivers power remotely through the skin or a battery. Alternatively, the force needed to expel the fluid can be provided by a collapsing, e.g., a soft-walled or resilient-walled elastomeric infusion pump. Such a pump/reservoir could be formed from, for example, rubber or plastic, similar to a Painbuster® pain management system from I-Flow Corp. In certain embodiments, the pump could be actuated by a pump actuator that is powered and/or controlled separately from actuator 60.

The pump can be set to deliver the desired composition at controlled, regular intervals, such as delivery of set amounts of bone morphogenic proteins just after widening the gap in the bone to promote rapid consolidation. In some embodiments, the pump can be externally powered or controlled (or both), in a fashion similar to that of the externally powered and controlled actuators described herein, to permit delivery of the composition at the discretion of the physician. For example, where an antibiotic is contained in the reservoir, the medication could be reserved until such time as it is indicated.

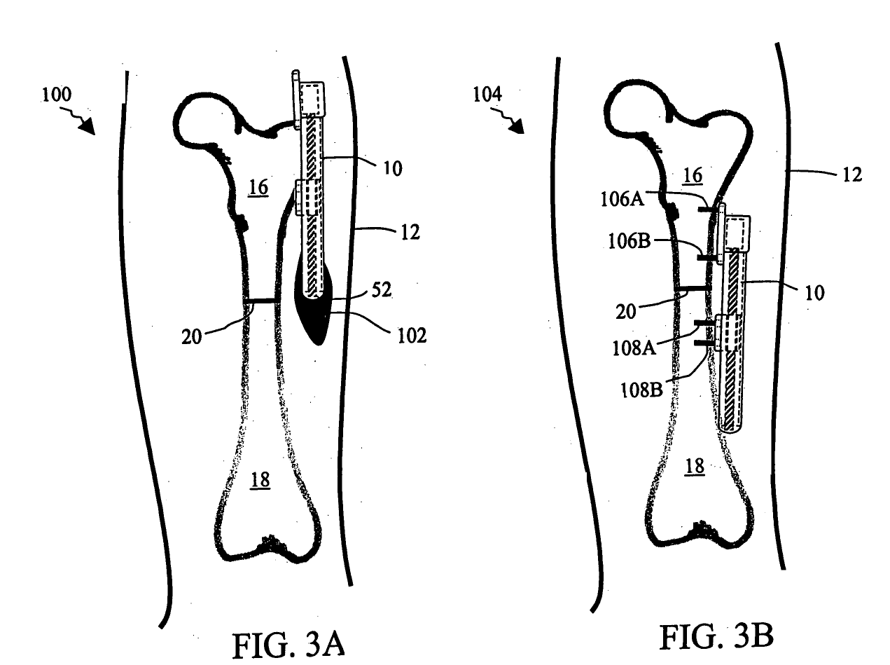

FIGS. 3A and 3B illustrate a method of implanting the extramedullary bone-lengthening devices into a leg and attaching the device to a femur. In a first step, the device 10 is implanted through an incision 102 using a minimally invasive technique. This step is done prior to, in conjunction with, or after an osteotomy or surgical division of the bone 14 at an osteotomy gap 20. This step can be done according to what is conventionally referred to as a bridge plating technique. The bridge plating technique will not violate the growth plates nor risk femoral head avascular necrosis (AVN), and is independent of intramedullary canal size, which are significant advantages of the new devices. The end 52 is inserted first through the incision 100. The smooth contour of the end cap 52 enables the device 10 to be inserted with minimal snags on the skin 12 and other tissue surrounding the bone 14. The smooth edges 58A, 58B, 58C, 58D (shown in FIG. 2B) as well as the smooth edges 70A, 70B, 70C, 70D (also shown in FIG. 2B) also minimize snags on the skin 12 and other tissue during insertion of the device 10.

Referring to FIG. 3B, in step 104, the device 10 is fastened to the bone parts 16, 18 using screws 106A, 106B, and others not shown through clearance holes 56A, 56B, 56C, 56D, respectively and screws 108A, 108B, and others (not shown) through clearance holes 68A, 68B, 68C, 68D (shown in FIG. 2B), respectively. In subsequent steps (not shown), the device 10 separates bone parts 16, 18 to increase the osteotomy gap 20 gradually while allowing bone tissue to grow in the osteotomy gap 20. After full consolidation of the bone, e.g., after 4, 6, 8, 10, 12 months or more, the screws 56, 58 and the device 10 may be removed through new incisions in the skin 12.

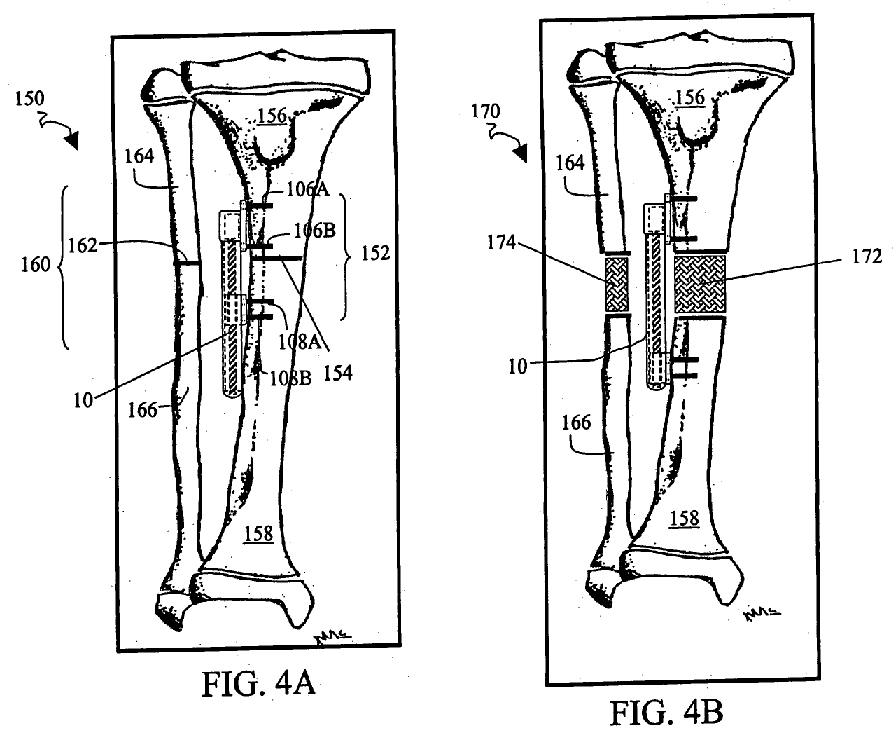

Referring to FIG. 4A, in an example of elongation of a tibia 152, step 150 includes performing an osteotomy 20 and implanting the device 10 using screws 106, 108 on the tibia 152. The osteotomy 20 separates the tibia 152 into parts 156, 158. Another osteotomy 162 is made on a fibula 160 separating the fibula 160 into parts 164, 166. The fibula 160 is connected via end tendons to the tibia 152.

Referring to FIG. 4B, step 170 includes actuating the device 10 to separate the tibia parts 156, 158. As the parts 156, 158 are pulled apart, the end tendons connecting the tibia 152 and the fibula 160 pull the fibula parts 164, 166 apart. New tissue 172 grows in the separation between the tibia parts 156, 158 as new tissue 174 grows in the separation between the fibula parts 164, 166. Subsequently, the device is removed from the tibia 152 as described previously.

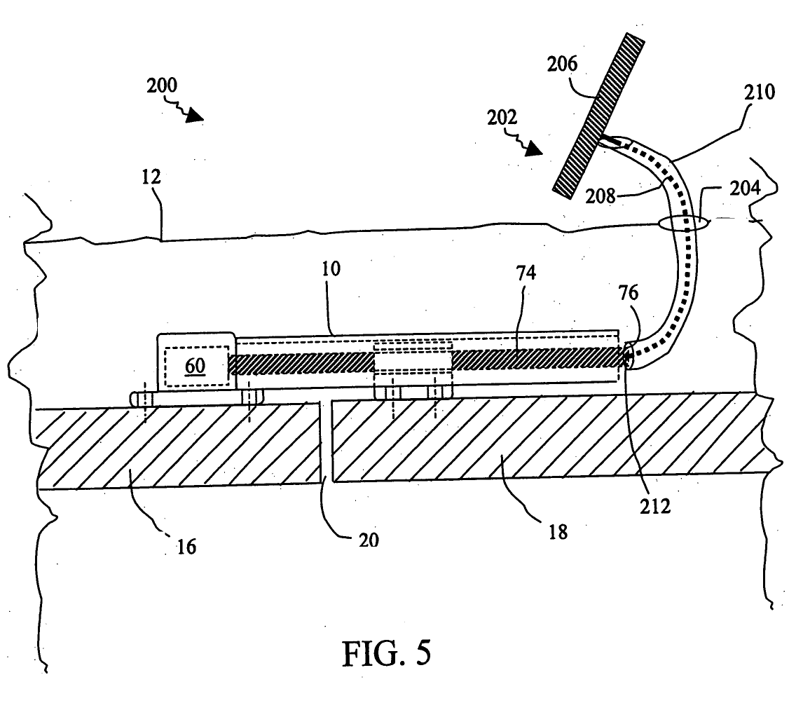

Occasionally, it may be necessary to manually actuate the device 10 after implantation. This can happen when there is premature bone consolidation or failure of the actuator 60. When there is premature bone consolidation, an extra force beyond the capacity of the actuator 60 may have to be applied to separate the two bone parts. This extra force can be supplied by manually actuating the device 10. In such cases, referring to FIG. 5, in configuration 200, the end cap 52 can be removed and a manual actuator 202 can be inserted through an incision 204. The manual actuator 202 includes a handle 206 attached to a flexible mechanical linkage 208. The linkage 208 is inside a sleeve 210. The end 212 of the linkage 208 makes a temporary mechanical connection with the end 76 of the threaded rod 74. In some examples, the end 212 is a socket driver that mates with a socket cavity at the end 76 of the threaded rod 74. In this configuration, after mating the end 212 to the end 76 of the threaded rod 74, a surgeon twists the handle 206 to turn the threaded rod 74 and to drive the actuator 60. The threaded rod 74 can be rotated in either direction depending on the therapeutic requirements for the bone 14.

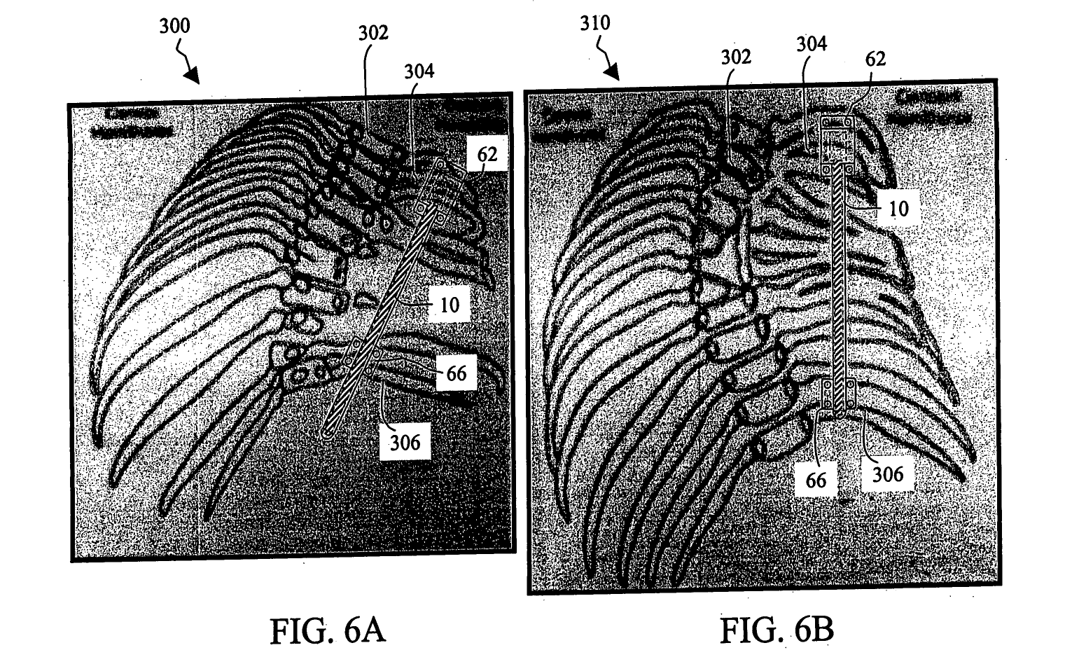

Referring to FIG. 6A, in a configuration 300, the device 10 can also applied to a spine for gradual elongation or correction of curvature. Configuration 300 is used primarily for children with infantile scoliosis that does not respond to conservative therapy, or other young children with severe kyphotic or scoliotic deformities and possible pulmonary compromise that are too young to fuse. In configuration 300, the device 10 is implanted under the skin such that the plate 62 is secured to one or more ribs 304 and the plate 66 is secured to one or more ribs 306. As the device 10 is actuated, the spine 302 is elongated and straightened as shown in configuration 310, in FIG. 6B. The device 10 moves the plates 62 and 66 apart at a rate of translation from about 0.25 to 2 millimeter (mm) per day. Subsequently, the device 10 can be removed from the ribs. In this embodiment, the device can be designed with a flat configuration to avoid protruding too much when implanted adjacent to the ribs. For example, the device may have a height of no more than 0.5 to 1.0 cm.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An extramedullary elongation device for lengthening one or more bones, the device to be implanted adjacent to the bone and under the skin of a patient using minimally invasive techniques, the device comprising:
a frame having smooth edges and an end with a smooth contour;
a first plate attached to the frame and configured to be secured to the bone, the first plate having smooth edges;
a second plate configured to be secured to the bone, the second plate having smooth edges;
a rod linked to the first plate;
an actuator secured either to the rod or the second plate; and
a block secured to the second plate, the block linked to the rod such that actuation of the actuator results in displacement of the second plate relative to the first plate; wherein the device has a cross-sectional diameter of less then about 3.0 cm.

2. The elongation device of claim 1, wherein the rod is threaded and enclosed by the frame, and the block comprises a threaded hole configured to be threaded on the rod.

3. The elongation device of claim 2, wherein the actuator is secured to the threaded rod and the first plate, the first plate is attached to the frame, actuation of the actuator rotates the rod, and the second plate is slideably engaged with the frame.

4. The elongation device of claim 2, further comprising a manual crank that is located externally to the skin of the patient, wherein the manual crank is configured to be mechanically connected to the rod through an opening in the skin such that rotation of the crank rotates the rod.

5. The elongation device of claim 1, wherein the actuator is attached to the second plate, the first plate is attached to the frame, and the second plate is slideably engaged with the frame.

6. The elongation device of claim 5, wherein the actuator is a linear positioning stage.

7. The elongation device of claim 1, wherein the actuator is a bi-directional motor, and wherein the elongation device further comprises a controller that controls the actuator.

8. The elongation device of claim 7, wherein the controller is located externally to the skin of the patient and the controller transmits power and control signals via radio frequency to the motor.

9. The elongation device of claim 1, wherein the first and second plates each have two or more holes, and the plates are each secured to the bone using two or more screws.

10. The elongation device of claim 1, wherein the controller is connected to the device.

11. The elongation device of claim 1, wherein the first and second plates are configured to be secured to a long bone such as a tibia or a femur, and wherein displacement of the second plate in a first direction relative to the first plate results in elongation of the long bone.

12. The elongation device of claim 1, wherein the first plate is configured to be secured to a first rib bone or vertebra and the second plate is configured to be secured to a second rib bone or vertebra, and the displacement of the second plate in a first direction relative to the first plate results in elongation or straightening of a spinal column.

13. The elongation device of claim 1, wherein the actuator is operative to displace the second plate relative to the first plate at a rate of from about 0.25 millimeter to 2 millimeters per day.

14. The elongation device of claim 1, wherein the actuator is powered by a spring.

15. The device of claim 1, further comprising a fluid reservoir and a conduit leading from the fluid reservoir to a space between the first and second plates.

16. The device of claim 15, further comprising a pump operative to pump a therapeutic fluid from the fluid reservoir through the conduit.

17. The device of claim 16, wherein the pump is actuated by the actuator.

18. The device of claim 16, wherein the pump is actuated by a pump actuator.

19. A method for elongating a long bone of a patient, the method comprising:
performing an osteotomy of the bone;
implanting the elongation device of claim 1 using a minimally invasive technique by making an incision in the skin, inserting the end of the frame into the incision, and then sliding the remainder of the device through the incision;
securing the first plate to the bone on one side of the osteomy;
securing a second plate to the bone on the opposite side of the osteomy; and
closing the incision.

20. The method of claim 19, further comprising actuating the actuator.

21. The method of claim 19, wherein the first and second plates each have two or more holes, and securing the device further comprises:
drilling four or more holes in the bone; and
placing screws through the holes in the plates and into the bone.

22. The method of claim 19, wherein the first and second plates of the device are configured to be secured to a long bone such as a tibia or a femur, and the translation of the second plate relative to the first plate results in elongation of the long bone.

23. The method of claim 19, wherein the actuator translates the second plate relative to the first plate from about 0.25 millimeter to 2 millimeters per day.

24. The method of claim 19, wherein the elongation device further comprises a fluid reservoir and a conduit leading from the fluid reservoir to a space between the first and second plates, the method further comprising:
filling the reservoir with a therapeutic fluid prior to implanting the device;
after implanting the device, delivering therapeutic fluid from the reservoir through the conduit to the space between the first and second plates of the device.

25. A method for straightening a spine of a patient, the method comprising:
creating an incision in the skin over a rib cage of the patient;
implanting the elongation device of claim I using a minimally invasive technique by inserting the end of the frame into the incision, and then sliding the remainder of the device through the incision;
securing the first plate to the first rib;
securing a second plate to the second rib; and
closing the incision in the patient.

26. The method of claim 25, wherein the elongation device further comprises a fluid reservoir and a conduit leading from the fluid reservoir to a space between the first and second plates, the method further comprising:
filling the reservoir with a therapeutic fluid prior to implanting the device;
after implanting the device, delivering therapeutic fluid from the reservoir through the conduit to the space between the first and second plates of the device.

27. An extramedullary elongation device for lengthening one or more bones, the device to be implanted adjacent to the bone and under the skin of a patient using minimally invasive techniques, the device comprising:
a first plate attached to the frame;
a second plate;
a rod linked to the first plate;
an actuator secured either to the rod or the second plate;
a block secured to the second plate, the block linked to the rod such that actuation of the actuator results in displacement of the second plate relative to the first plate; and
screws for securing the first plate and second plate to a bone, wherein the screws are no more than about 50 mm in length.

28. The device of claim 27, wherein the screws are no more than about 40 mm in length.

29. The device of claim 27, wherein the screws are threaded for substantially their entire length.

30. The device of claim 27, wherein the first plate and second plate each have two or more threaded holes for receiving the threads of the screws.

31. The device of claim 27 further comprising a frame attached to the first plate and enclosing the rod, wherein the first plate and second plate have smooth rounded edges and the frame has smooth rounded edges and an end with a smooth contour.

32. The device of claim 27, further comprising a fluid reservoir and a conduit leading from the fluid reservoir to a space between the first and second plates.

33. An elongation device for lengthening one or more bones, comprising:
an actuator;
a first member configured to be secured to the bone;
a second member configured to be secured to the bone and operably connected to the first member such that actuation of the actuator results in displacement of the second member relative to the first member;
a fluid reservoir connected to a conduit; and
a pump operative to pump fluid from the fluid reservoir through the conduit upon actuation of the actuator.

34. The device of claim 33, wherein the reservoir further contains an inlet port for filling the reservoir.

35. The device of claim 34, wherein the inlet port is covered by a septum.
